(19) United States
(12) Patent Application Publication
(10) Pub. No.: US 2010/0093652 A1
(43) Pub. Date: Apr. 15, 2010

(54) Preservative System for Emulsion-Based Therapeutic Topical Formulations

(75) Inventors: Nicholas Spring, Jenkintown, PA (US); Edward J. Delaney, Princeton, NJ (US)

Correspondence Address: RATNERPRESTIA, P.O. BOX 980, VALLEY FORGE, PA, 19482, US

(73) Assignee: Topaz Pharmaceuticals Inc., Horsham, PA (US)

(21) Appl. No.: 12/608,345

(22) Filed: Oct. 29, 2009

Related U.S. Application Data
(63) Continuation-in-part of application No. 12/115,291, filed on May 5, 2008.
Application No. 12/115,291 is a continuation-in-part of application No. 11/871,660, filed on Oct. 12, 2007.
(60) Provisional application No. 61/109,325, filed on Oct. 29, 2008.
(60) Provisional application No. 60/976,259, filed on Sep. 28, 2007.
(60) Provisional application No. 60/916,107, filed on May 4, 2007.
(60) Provisional application No. 60/976,259, filed on Sep. 28, 2007.
(60) Provisional application No. 60/851,352, filed on Oct. 12, 2006.

(51) Int. Cl. A61K 31/7048; A01N 43/14; A01P 1/00; A61P 33/00

(52) U.S. Cl. 514/32

(57) ABSTRACT

A topical formulation that inhibits the growth of microorganisms comprises an effective amount of an insecticide dissolved in an oil phase comprising a water-miscible or water-soluble surface active agent, a suspending agent, and a non-ionic surfactant, and an aqueous phase comprising one or more preservatives, where the aqueous phase is buffered to a specific pH. The overall antimicrobial efficacy in an emulsion formulation can be enhanced by buffering the aqueous phase of the emulsion to a pH at which the preservative system inhibits growth for a wider variety of microorganism types in the formulation relative to the equivalent formulation in which the aqueous phase is not buffered.

### CROSS REFERENCE TO RELATED APPLICATIONS

This application claims priority to U.S. Provisional Application No. 61/109,325, filed Oct. 29, 2008, and is a continuation-in-part of U.S. patent application Ser. No. 12/115,291 filed May 5, 2008, which claims priority to U.S. Provisional Application Nos. 60/976,259, filed Sep. 28, 2007, and 60/916,107, filed May 4, 2007, and is a continuation-in-part of U.S. patent application Ser. No. 11/871,660, filed Oct. 12, 2007, which claims priority to U.S. Provisional Application Nos. 60/976,259, filed Sep. 28, 2007, and 60/851,352, filed Oct. 12, 2006. Each of these applications is incorporated herein by reference, in its entirety and for all purposes.

### FIELD OF THE INVENTION

The invention relates generally to the field of formulation chemistry. More particularly, the invention relates to improved preservative systems for emulsion-based therapeutic topical formulations, especially improved preservative systems for emulsion-based avermectin formulations. The invention further relates to topical emulsions in which the aqueous phase is buffered within an acidic pH range within which the preservative system provides a product shelf life significantly longer than the same emulsion-based product without buffer, and, when the therapeutic active is an avermectin, base-catalyzed isomerization is also inhibited.

### BACKGROUND OF THE INVENTION

Emulsion formulations serve as useful vehicles for topical delivery of therapeutic actives. Emulsions, which combine oil and water phases in a single fluid having a monolithic appearance, have several advantages not offered by water-based or oil-based topical therapeutic compositions. First, by combining oil and water phases, emulsions make it possible to formulate hydrophilic and hydrophobic ingredients in a single composition. Second, despite having an oil component, emulsions appeal to consumers because they do not have an oily feel as palpable as purely oil-based formulations.

Third, and perhaps most important to topical therapeutics, emulsions have a rheology wherein they are stable at rest but exhibit a decreasing viscosity as an increasing shear stress is applied. This allows for ease of topical application, but, once applied, the emulsion does not run or drip, and remains in the area where applied that requires therapeutic treatment.

This is particularly advantageous with topical compositions for the treatment of head lice. The product must remain securely where applied for a period of time effective to eradicate the lice. A watery product that quickly runs down the forehead and neck is both unpleasant to apply and ineffective.

Emulsions should remain stable to retain their desirable rheological properties. Separation of the oil and water components will result in a drop in viscosity and produce a drippy, runny product with an oily feel that will not remain in place when applied. Emulsion-based topical therapeutic products should remain stable over their intended useful life.

The therapeutic active must also remain stable. Avermectins are known to undergo base-catalyzed isomerization to form a 2-epimer impurity that has substantially reduced biological activity in comparison to the base product.

Furthermore, many oils and other emulsion components suitable for topical application serve as a nutritional base for microorganisms such as fungi and bacteria. If unimpeded, microbial growth in the formulation may pose a risk of infection to a patient who has been bitten by head lice. In addition, the resulting microbial biomass has an unpleasant odor and appearance, and may otherwise make the product unsuitable for skin contact. There is a need for improvement to the preservative systems of emulsion formulations.

### SUMMARY OF THE INVENTION

The invention features topical formulations resistant to the growth of microorganisms, including bacteria, yeast, fungi, molds, and the like. The formulations generally comprise an effective amount of an insecticide dissolved in an oil phase comprising a water-miscible or water-soluble surface active agent, a suspending agent, and a non-ionic surfactant, and an aqueous phase comprising one or more preservatives and which is buffered to a pH at which the formulation is resistant to microorganism growth to a greater extent or is otherwise microbicidal relative to the equivalent formulation in which the aqueous phase is not buffered or buffered differently.

The insecticide preferably comprises an avermectin such as ivermectin, doramectin, selamectin, or abamectin, or combinations thereof. Ivermectin is preferred. The insecticide can further comprise a spinosyn, such as spinosyn factors A, B, C, D, E, F, G, H, J, K, L, M, N, 0, P, Q, R, S, T, U, V, W and Y, or combinations thereof. Spinosad is a preferred spinosyn. Ivermectin can be included at a concentration of about 0.1 to about 1% by weight of the formulation. Where ivermectin and spinosad are used, they can be present at a combined concentration of about 0.1% to about 5% by weight of the formulation.

The pH is preferably buffered in the range of about 4.5 to about 6.2. The buffering agent preferably is or comprises citric acid and/or sodium citrate, which may be added to a concentration of about 0.01% to about 0.1% citric acid and from about 1.0% to about 1.25% sodium citrate, by weight of the formulation.

The oil phase can comprise 20% to 35% of the suspending agent by weight of the formulation, and the suspending agent can comprise olive oil, shea butter, or combinations thereof. The olive oil can comprise 25% to 28% by weight of the formulation, and the shea butter can comprise 1% to 5% by weight of the formulation. The oil phase can comprise 15% to 45% of the non-ionic surfactant by weight of the formulation, and the non-ionic surfactant can comprise oleyl alcohol, lanolin alcohol, sorbitan tristearate, or combinations thereof. The oil phase can comprise 10% to 20% of the water-miscible or water-soluble surface active agent by weight of the formulation, and the water-miscible or water soluble surface active agent can comprise polysorbate 80, cetyl acetate, acetylated lanolin alcohol, or combinations thereof.

The formulation preferably comprises a preservative such as methylparaben and propylparaben, which can be present in the formulation at a combined concentration of 0.01 to 2% by weight of the formulation.

The formulation can comprise a conditioner such as cyclomethicone. The conditioner can be present at a concentration of 1% to 5% by weight of the formulation.

The invention also features methods for enhancing the resistance of a topical emulsion formulation to microbial growth, including bacterial, fungal, mold, and/or yeast growth. The methods generally comprise buffering the aqueous phase of an emulsion to a pH at which a greater amount of microorganism growth in the formulation is inhibited relative to the equivalent formulation in which the aqueous phase is not buffered or buffered to a more acidic, neutral, or basic pH. Preferred emulsions include those described herein.

The emulsion can be buffered to a pH in a preferred range of from about 4.5 to about 6.2. The aqueous phase of the emulsion can be buffered using a reagent comprising an effective amount of citric acid and/or sodium citrate. Preferably, when the methods are employed using a formulation comprising ivermectin, the pH also or alternatively inhibits base-catalyzed isomerization of the ivermectin.

The invention also features methods for treating a head lice infestation from a susceptible or treatment-resistant strain of head lice in a subject, such as a human. Generally, the methods comprise topically administering to the subject an effective amount of an emulsion formulation as described herein for a period of time sufficient to treat the head lice infestation. Preferably, the formulation is topically administered to the subject in a single dose. The period of time can range from about 1 minute to about 60 minutes, although shorter or longer periods of time can be used. If multiple administrations are employed, the interval between each dose is preferably from 5 to 9 days.

### DETAILED DESCRIPTION OF THE INVENTION

Various terms relating to the systems, methods, and other aspects of the invention are used throughout the specification and claims. Such terms are to be given their ordinary meaning in the art unless otherwise indicated.

As used in this specification and the appended claims, the singular forms “a”, “an,” and “the” include plural referents unless the content clearly dictates otherwise.

The term “about” as used herein is meant to encompass variations of ±20% or ±10%, more preferably ±5%, even more preferably ±1%, and still more preferably ±0.1% from the specified value.

It has been observed in accordance with the invention that antimicrobial performance in emulsion formulations is enhanced by buffering the aqueous phase to a pH of about 4.5 to about 6.2. Accordingly, the invention features topical emulsion formulations, including those comprising preservatives, in which the resistance of the formulations to microbial growth is enhanced when the aqueous phase of the emulsion is buffered accordingly, relative to similar formulations in which the aqueous phase is not buffered, or buffered to a more acidic, neutral, or basic pH. The invention also features methods for enhancing the microbial resistance and/or antimicrobial properties of topical emulsions.

Topical formulations according to the invention can be aqueous dispersions of an oil phase containing a therapeutically active ingredient. The aqueous dispersions may be prepared as water-in-oil emulsions or oil-in-water emulsions, depending upon the rheological properties desired by the product formulator. How the disclosed ingredients of the invention may be formulated as water-in-oil or oil-in-water emulsions is well understood by one of ordinary skill in the formulation of personal care products such as shampoos and hair conditioners.

The formulations of the invention are particularly well-suited for the topical application of a therapeutically active ingredient that is hydrophobic and poorly solvated by water. Thus, the therapeutically active ingredient preferably is dissolved in suitable agents to improve the stability of the active ingredient in water. Preferably, these agents are easily soluble in water or are water miscible, and include, for example, surfactants, including water-soluble or water-miscible surface active agents. Water-soluble and water-miscible surface active agents include compounds that can dissolve a therapeutically active ingredient and stabilize the active ingredient in water. Highly preferred topical emulsions comprise both an oil phase and an aqueous phase, with the active ingredient dissolved in the oil phase. The oil phase preferably comprises three categories of ingredients: a solubilizer, a non-ionic surfactant, and a suspending agent.

### Active Agents

The active agents are preferably pesticides or insecticides, and more preferably insecticides capable of killing lice.

One aspect of the invention relates to topical formulations containing avermectins that are useful as agents in the treatment or prevention of an infestation of head lice, where the infested lice may be either a susceptible- or treatment-resistant strain of Pediculus humanus capitis. Treatment-resistant strains include those resistant to one or more pesticides/insecticides, and particularly those ordinarily prescribed or indicated for treating lice infections. Suitable avermectins include ivermectin, doramectin, selamectin, avermectin B₁ₐ, avermectin B_{1b}, selamectin, eprinomectin, and abamectin, with ivermectin being highly preferred.

One or more avermectins may be present in the formulations at varying concentrations, for example, from about 0.005% to about 5% by weight of the formulation. For example, a 1% avermectin formulation would include 1 gram (g) avermectin per 100 milliliters (ml) of formulation volume.

In some aspects, the avermectin is present at a concentration of about 0.1% to about 2% by weight of the formulation. It has been observed in accordance with the present invention that ivermectin concentrations of 0.25%, 0.5% and 1% promote effective killing of a permethrin-resistant strain of head lice. Ivermectin can be utilized as a mixture of over 80% 22,23-dihydroavermectin B₁ₐ and less than 20% 22,23-dihydro-avermectin B_{1b} and preferably a mixture of at least 90% 22,23-dihydroavermectin B₁ₐ and less than 10% 22,23-dihydroavermectin B_{1b}. An avermectin such as ivermectin can be dissolved in the formulation at about 0.05% to 5% by weight, about 0.1% to 2% by weight, or about 0.25% to 1% by weight, for example.

The topical formulations can comprise combinations of at least one avermectin and at least one spinosyn. The spinosyns can be present at varying concentrations at a weight/volume percentage of, for example, from about 0.005% to about 5% by weight of the formulation. A spinosyn can be dissolved in the formulation at about 0.05% to 5% by weight, about 0.1% to 5% by weight, about 0.1% to 2%, or about 0.25% to 1% by weight, for example.

Spinosyns include, but are not limited to, individual spinosyn factors A, B, C, D, E, F, G, H, J, K, L, M, N, 0, P, Q, R, S, T, U, V, W, or Y, and any combinations thereof. Spinosad refers to a combination of the spinosyn factors spinosyn A and spinosyn D, where spinosyn A comprises approximately 85% and spinosyn D comprises approximately 15% of the spinosad.

Combinations of spinosyns and avermectins can comprise 0.01 to about 5% of the formulation weight, preferably about 0.1 to about 5% by weight, preferably 0.25% to about 2% by weight, and preferably 0.5% to 3% by weight of the formulation.

It is highly preferred that the insecticide or combination of insecticides is dissolved and remains dissolved in the oil phase of an emulsion formulation. It is preferable that the active agent does not precipitate from solution.

### Solubilizers

The oil phase of the emulsion can comprise one or more solubilizers. Solubilizers can comprise a water-soluble or water miscible surface-active agent, or combinations thereof. The water-soluble or water-miscible surface active agent can comprise at least about 10% of the formulation weight. In some aspects, this agent comprises about 10% to about 50% of the formulation weight, and in some aspects, this agent comprises about 20% to about 50% of the formulation weight. In highly preferred aspects, this agent comprises about 10% to about 20% of the formulation weight.

Any suitable water-soluble or water-miscible surface active agent can be used. Non-limiting examples include polysorbate 80, cetyl acetate, and acetylated lanolin alcohol, or combinations thereof. Crodalan AWS, available from Croda Chemicals, is a suitable reagent the comprises polysorbate 80, cetyl acetate, and acetylated lanolin alcohol.

Polysorbate 80 can be present in the formulation at about 5% to about 25% by weight, from about 10% to 15% by weight, and preferably about 11.25% to 13.5% by weight of the formulation. Cetyl acetate can be present in the formulation at about 0.5% to 10% by weight, from about 1% to 4% by weight, and preferably about 1.50% to 3.75% by weight of the formulation. Acetylated lanolin alcohol can be present in the formulation at about 0.10% to 3% by weight, from about 0.5% to about 1% by weight, and preferably 0.15% to 0.75% by weight of the formulation.

In some aspects, having the water-soluble or water-miscible surface active agent bound to the surface of the therapeutically active ingredient can ensure that the active ingredient is stable in the aqueous environment of the emulsion. In some aspects of the invention, the therapeutically active ingredient may be stabilized by pharmaceutically accepted glycols present in the formulation at a level below 30% by weight, such as, for example below 25%, or below 20% or below 15% or below 10% or below 5%. In some highly preferred aspects, the formulation does not include any glycols, especially propylene or polyethylene glycol.

### Suspending Agents

The oil phase of the emulsion can comprise one or more suspending agents. In some aspects, a combination of fatty oils and/or fats can serve as a suspending agent. Examples of suspending agents include, without limitation, olive oil, shea butter, coco butter, vegetable oil, and the like. Olive oil is a triacylglyceride, where three fatty acids are tethered to a glycerol backbone, and shea butter is primarily made of palmitic, stearic, oleic, linoleic, and arachidic fatty acids. Although these fatty acids have been used as “home remedies” for removal of head lice from the scalp, they do not kill head lice. Both olive oil and shea butter are viscous materials that slow the movement of adult lice to better remove them. Combinations of olive oil and shea butter are preferred suspending agents.

In some aspects, olive oil is present in the formulation at a level of about 20% to 30% by weight of the formulation, and preferably from about 25% to 28% by weight (e.g., about 27.5% by weight). Shea butter can be present in the formulation at a level of about 1% to 5% by weight of the formulation, and preferably about 2% by weight. Other known suspending agents which can be utilized in the formulations and related methods include, but are not limited to, coconut oil, palm oil, cottonseed oil, vegetable oil, soybean oil, olive oil, peanut oil, corn oil, sunflower oil, safflower oil, jojoba oil, canola oil, shea butter, cocoa butter, milk fat, amaranth oil, apricot oil, argan oil, avocado oil, babassu oil, ben oil, alga-roba oil, coriander seed oil, false flax oil, grape seed oil, hemp oil, kapok seed oil, meadowfoam seed oil, okra seed oil, perilla seed oil, poppy seed oil, prune kernel oil, pumpkin seed oil, quinoa oil ramtil oil, rice bran oil, camellia oil, thistle oil, wheat germ oil and combinations thereof. Fatty acid glycerides can be used and have known use as skin moisturizers.

### Non-Ionic Surfactants

The oil phase of the emulsion can comprise one or more non-ionic surfactants. Non-ionic surfactants include compounds that act at the water-air and water-oil inter-faces thereby enhancing wetting ability, emulsion stabilization, foaming, rheology, antistatic, lubricity and surface conditioning properties of the emulsion. In some aspects of the invention, a fatty alcohol or a mixture of fatty alcohols can serve as non-ionic surfactants. Apart from additional stabilization of the active ingredient, the non-ionic surfactants have various purposes in the surface chemistry of the formulation, when the formulation is used in a final product such as a body wash or a shampoo-conditioner.

In addition to their surface-active properties, fatty alcohols are emollients that make the skin smoother and act at the water-air and water-oil interfaces, thereby enhancing wetting ability, emulsion stabilization, foaming, rheology, antistatic, lubricity, and surface conditioning properties of the formulation. Emollients include compounds that soften and smooth skin by preventing the skin from losing moisture. Examples of suitable non-ionic surfactants include, without limitations, oleyl alcohol, lanolin alcohol, sorbitan tristearate, bees wax, erucyl alcohol, ricinolyl alcohol, arachidyl alcohol, capryl alcohol, capric alcohol, behenyl alcohol, lauryl alcohol, myristyl alcohol, cetyl alcohol, stearyl alcohol, isostearyl alcohol, oleyl alcohol, palmitoleyl alcohol, linoleyl alcohol, elaidyl alcohol, elaidolinoleyl alcohol, linolenyl alcohol, elaidolinolenyl alcohol, and combinations thereof.

In some preferred aspects, the formulation includes non-ionic surfactants at a combined concentration of about 10% to 35% by weight of the formulation, or preferably about 15% to about 24% by weight, and more preferably about 18-24% by weight. Preferred non-ionic surfactants comprise oleyl alcohol, lanolin alcohol, and sorbitan tristearate. In some aspects, oleyl alcohol can be present in the formulation at about 5% to 15% by weight of the formulation, and preferably about 10% by weight. Lanolin alcohol can be present in the formulation at about 3% to about 15% by weight, more preferably from about 5% to 10% by weight, and more preferably about 8% by weight. Sorbitan tristearate is available commercially as Glycomul® TS (Lonza, Inc.) or SPAN 65 as sold by Merck Schuchardt OHG. Sorbitan tristearate is a low HLB ester based surfactant and has many uses in the food and cosmetic industries. The chemical structure of sorbitan tristearate is defined by a cyclic five member ether, with hydroxyl groups, and three fatty acid side chains. Sorbitan tristearate can be present in the formulation at about 0.1% to 3% by weight of the formulation, preferably about 0.5% by weight.

### Conditioners

A silicone compound can be added to the formulation, in some aspects, preferably to the oil phase of the emulsion, to serve as a skin or hair conditioner. Conditioning agents can change the texture, feel and appearance of human hair. Conditioning agents other than silicone compounds may also be used. In some aspects, the silicone compound can be selected from volatile silicones, of which cyclomethicone is one. Cyclomethicone can act as a conditioner in formulations to be applied to the hair, such as shampoo-conditioners. It gives a soft, silky feel to hair and evaporates quickly leaving little residue. Cyclomethicone can be included in formulations at about 1% to 5% by weight of the formulation, and preferably at about 3% by weight. Examples of conditioners that can be used include, but are not limited to, cyclomethicone, dimethicone, hexamethyldisiloxane, octamethyltrisiloxane, decamethyltetrasiloxane, dodecamethylpentasiloxane, polydimethylsiloxanes, and combinations thereof.

### Preservatives

The formulations preferably comprise a preservative to inhibit the growth of microorganisms and/or to protect the formulation from chemical breakdown. In some aspects, preservatives can be selected from compounds in the paraben family. Preferred parabens include methylparaben, propylparaben, or combinations thereof. The preservatives are preferably water-soluble, and preferably are comprised in the aqueous phase of the emulsion.

In some aspects, the combined concentration of preservatives in the formulation is about 0.05% to about 2% by weight of the formulation. Methylparaben can be present in the formulation at about 0.01% to 2% by weight of the formulation, and preferably about 0.1 to about 0.3% by weight, and more preferably about 0.20% by weight. Propylparaben can be present in the formulation at about 0.01% to about 1% by weight, and more preferably about 0.01% to about 0.5% by weight, more preferably about 0.05% to about 0.1% by weight, and more preferably about 0.1% by weight. Other suitable preservatives include, but are not limited to, methylparaben, propylparaben, ethylparaben, butylparaben, isobutylparaben, isopropylparaben, benzylparaben, and salts thereof.

### pH Modifiers and Buffering Agents

The formulation preferably has a pH of about 1.0 to about 6.5. A pH between about 4.0 and about 6.5 is preferred, a pH between about 4.5 and about 6.2 is preferred, a pH between about 4.5 and 6.0 is preferred, a pH between about 5.0 and about 6.0 is more preferred, and a pH between about 5.3 and about 5.8 being even more preferred. Highly preferred is the pH of human skin. Buffers capable of buffering the aqueous phase to a pH between about 4.5 and about 6.2 are commonplace and readily identified by those of ordinary skill in the art. Such buffers include, for example, citrate buffer, acetate buffer, phosphate buffer, tartrate buffer, fumarate buffer, dimethylglutarate buffer, succinate buffer, phthalate buffer, maleate buffer, and mixtures thereof. The agent preferably comprises a biologically acceptable carboxylate that is capable of buffering an aqueous phase of an emulsion, non-limiting examples of which are described in the preceding list.

Combinations of citric acid and sodium citrate are highly preferred. pH modifiers can be mixed directly into the formulation in their solid form, or can be added as part of a liquid or solid buffer comprising multiple agents.

In some aspects, citric acid is included in the formulation at about 0.01% to about 0.1% by weight of the formulation, and preferably about 0.055% by weight. In some aspects, these concentrations of citric acid are combined with about 1% to about 1.25% sodium citrate by weight of the formulation, preferably about 1.099% by weight of citric acid. The amount of citric acid and/or sodium citrate can vary according to the desired pH to be achieved with the formulation. In highly preferred aspects, about 0.055% by weight citric acid is used in combination with about 1.099% or about 1.1% by weight sodium citrate. These agents can themselves provide antimicrobial activity independent of the preservatives, and in some aspects may be used in lieu of a separate preservative.

Sodium phosphates and/or sodium mono- and di-hydrogen phosphates can also be used in combination with citric acid and/or sodium citrate. Other suitable buffering agents include those comprising acetic acid and/or sodium acetate.

In some aspects where an avermectin is used as the therapeutically active ingredient, the buffer can also serve to prevent isomerization of the avermectin to the 2-epimer.

Other suitable preservatives include sodium benzoate, imidazolidinyl ureas such as the GERMALL family of preservatives, including GERMALL PLUS, polyvalent chelating agents such as EDTA and related compounds in various stages of protonation, and citric acid and sodium salts thereof, either alone, or in combinations of sodium salt and free acid. The other suitable preservatives can also be present in the formula in the same concentration range as the parabens, e.g., at levels of about 0.01% to 2%, or alternatively from about 0.01% to about 0.5% by weight (e.g., 0.05% by weight).

### Humectants

In some aspects, the formulation can further comprise a humectant. Humectants are hygroscopic materials intended to prevent the formulation from drying out during the course of use and prior to rinsing from the skin, hair or scalp. The humectant may also function as a moisturizer in shampoo and conditioner formulations. Humectants are usually molecules with several hydrophilic groups, such as hydroxyl, amine, carboxylic acid groups and esters thereof that provide the molecule the ability to form hydrogen bonds with water molecules.

In some aspects, other components of the formulation serve a dual role as the humectant, such as many of the non-ionic surfactants, including, but not limited to oleyl alcohol, lanolin alcohol, acetylated lanolin alcohol, and the like. In another embodiment, the humectant is chosen from glycerine, glyceryl triacetate, sorbitol, xylitol, maltitol, polydextrose, quillaia, lactic acid, urea, and the like, and mixtures thereof.

### Water

The oil phase mixture comprising the dissolved therapeutically active ingredient, suspending agents, solubilizer, and non-ionic surfactants, can be dispersed in water, or the water can be dispersed in the oil phase In some aspects, the water is deionized. Water acts as a carrier and may be included as warranted for any respective formulation. In the exemplified formulation, water can be present in the formulation at about 30% to 40% by weight of the formulation, and preferably about 30% to about 33% by weight, and preferably about 32%.

The addition of the oil phase comprising the therapeutically active ingredient to the deionized water can result in a colloidal suspension of the active ingredient, where micelles form around the active ingredient and are arranged such that hydrophilic heads of the surfactants are in contact with the solvent water molecules and the hydrophobic tails of the surfactants are in contact with the active ingredient. This formulation is especially suitable for delivering therapeutically active ingredients in body washes and shampoo-conditioners, which, for shampoo-conditioners, gives the products a suitable washout and flow behavior, leaving the hair in good condition.

The inventive formulations invention is exemplified by, but not limited to, a topical emulsion formulation as disclosed in Table 1. This formulation is in the consistency of a shampoo-conditioner or cream and at least comprises an effective amount of a therapeutically active ingredient, as well as a solubilizing agent(s), water, a suspending agent(s), a surfactant(s), silicone compound(s), and a preservative(s), in any combination and/or concentration which may be contemplated by the artisan upon review of this specification

A specific therapeutically active ingredient concentration of 0.50% (w/v) is shown in Table 1. This concentration range is presented to exemplify the invention and not to limit the effective range that may be utilized by the artisan to practice the claimed invention.

TABLE 1

| Ingredient | % (by weight) |
|---|---|
| Therapeutic Active | 0.50 |
| Deionized Water USP | 31.75 |
| Olive Oil NF | 27.75 |
| Crodalan AWS | 15 |
| Citric Acid USP | 0.055 |
| Sodium Citrate USP | 1.099 |
| Oleyl Alcohol NF | 10 |
| Lanolin Alcohol NF | 8 |
| Cyclomethicone NF | 3 |
| Shea Butter | 2 |
| Sorbitan Tristearate | 0.50 |
| Methylparaben NF | 0.25 |
| Propylparaben NF | 0.10 |

Adjustments may easily be incorporated with components, known equivalent components, combinations of components, and respective concentrations to provide alternative formulations for uses disclosed herein.

Thus, the artisan will be aware that the percentage by weight of any component may be adjusted to compensate for the concentration of the active ingredient, the texture or rheology of the topical formulation and whether it is formulated as a water-in-oil or oil-in-water emulsion (e.g., shampoo, cream, gel) and that components may be added at differing concentrations or may be left out of a formulation or substituted with an equivalent component so as to provide for a topical formulation similar to the exemplified topical formulation described herein

The skilled artisan will appreciate that other beneficial agents can be added into a formulation of the instant invention. Such beneficial agents include, without limitation, vitamins, hair dyes, nutrients, anti-dandruff agents and the like. The artisan can properly select the beneficial agent or the combination thereof such that the at least one beneficial agent would not negate the beneficial aspects of the formulation.

The invention also features methods for enhancing the resistance of topical emulsion formulations, including the formulations described and/or exemplified herein, to microbial growth, or otherwise for enhancing the antimicrobial properties of the formulation. The methods generally comprise buffering the emulsion to a pH at which the preservatives inhibit a greater amount of microorganism growth in the formulation relative to an equivalent or to the same formulation in which the pH has not been buffered, or buffered to a different pH. The formulations exhibit an enhanced resistance to growth of bacteria, yeast, fungi, and molds, among other things.

The pH can be adjusted to a basic, neutral, or acidic level according to the formulation or according to the particular preservative being used. It is preferred that the pH be buffered to an acidic pH. In some aspects, the aqueous phase is buffered to a pH from about 4.5 to about 6.2, although a higher or lower pH can be used. The aqueous phase can be buffered to a pH from about 5 to about 6, or any other pH described herein.

The methods may comprise adding an effective amount of citric acid and/or sodium citrate to the aqueous phase of the emulsion. Such an effective amount can vary, and preferably is sufficient to bring the aqueous phase of the formulation to a pH level that enhances the overall antimicrobial efficacy of the formulation. By way of example but not of limitation, the aqueous phase can comprise from about 0.01% to about 0.1% citric acid and from about 1% to about 1.25% sodium citrate by weight of the formulation. These agents can themselves provide antimicrobial activity independent of the preservatives, and in some aspects may be used in lieu of a preservative.

In some aspects, the methods are applied to inhibit base-catalyzed isomerization of the active ingredient. Thus, the formulations can be buffered to inhibit isomerization of active ingredients, such as avermectins. For example, where the formulation comprises ivermectin, the methods can comprise buffering the formulation to a pH sufficient to inhibit base-catalyzed isomerization of the ivermectin. For complex liquids, such as the formulations described and exemplified herein, it was previously unknown whether buffering the pH of the aqueous phase could affect the stability of components in the oil phase, in view of the transient exposure of the oil phase components to the aqueous phase. Because basic pH can induce isomerization of avermectins (Pivnichny, J V et al. (1988) J. Agric. Food Chem. 36:826-8), it is now believed that acidifying the pH of the aqueous phase of an emulsion comprising an avermectin dissolved in the oil phase will prevent or reduce isomerization of the avermectin. Thus, preferably, the aqueous phase is buffered. The pH sufficient to inhibit base-catalyzed isomerization of the active may vary according to the particular active used. In the case of ivermectin, for example, the aqueous phase is preferably buffered to a pH from about 4.0 to about 6.5, including the pH values described and exemplified herein, and is more preferably buffered to the pH of human skin.

The topical formulations can, for example, be prepared by separately preparing the oil phase and aqueous phase, for example, by mixing together the ingredients for each phase, and then by mixing the oil and aqueous phases together according to any means suitable in the art. Various conditions attendant to mixing the oil and aqueous phases together (e.g., temperature, rate of heating and cooling, and the like) can be varied in order to ensure proper dispersion and stability of the resultant emulsion.

The invention further includes methods for prophylaxis or elimination of either susceptible or treatment-resistant head lice. The resistance can be resistance to any head lice treatments currently marketed or otherwise known in the art, such as malathion resistance, lindane resistance, pyrethrum resistance, or permethrin resistance. The methods utilize formulations comprising avermectin with or without a spinosyn in a topical formulation, including any formulations described or exemplified herein.

Generally, the methods comprise topically administering to a subject in need of such treatment an effective amount of the a topical emulsion formulation comprising an aqueous phase buffered to a pH capable of enhancing the antimicrobial properties of the formulation, or of preservatives in the formulation for a period of time sufficient to treat and preferably eliminate the head lice infestation. It is highly preferred that the formulation be administered to the subject in a single dose, although multiple doses can be used as necessary based on the determination of the subject or a medical practitioner, including two, three, four, or more doses.

The formulation, which can be a shampoo-conditioner, can be used once or twice during about a seven day period (e.g., day 1 and between about day 5 and day 9), as well as three or four times (with an initial application on day 1 followed for a second, third or fourth application at intervals from about 5 days to about 9 days). At each dosing, the formulation can be applied and remain on the site of infestation, such as the scalp, for from about 1 minute to about 60 minutes or from about 3 minutes to about 30 minutes, then rinsed with warm water. The formulation can also be allowed to remain on the site of infestation, such as the scalp, from about 5 minutes to about 20 minutes, and from about 10 minutes to about 15 minutes. About 10 minutes is highly preferred. The shampoo-conditioner is preferably formulated to leave the hair in good condition while ridding the scalp of lice.

The methods can be applied to any animal, including companion animals and farm animals. Human beings are most preferred.

Dosing can be varied either by altering the avermectin or spinosyn concentration, as noted above, or by increasing the amount of topical formulation applied to the scalp of the human subject. While an ivermectin and spinosad based formulation is useful to practice the invention, other known avermectins beside ivermectin and other known spinosyns beside spinosad are also contemplated and have utility as the active ingredient component for the invention.

While dosing ranges may vary, a single application (dosage) to the scalp of an ivermectin/spinosad containing formulation of the invention preferably can range from about 1 ml to about 200 ml. In some preferred aspects, the dose is from about 3 ml to about 75 ml, more preferably from about 50 ml to about 120 ml, and more preferably from about 100 ml to about 120 ml. The dose amount can vary, for example, according to the amount and/or length of the hair. Thus, for example, longer hair may necessitate a larger dose.

In some aspects, at least about 60 ml of the topical formulation is applied to totally saturate the roots and to effectively cover the entire scalp area. The practitioner can vary the ivermectin and spinosad concentrations and/or volume of the topical formulation to manipulate the effective amount of ivermectin and spinosad to be administered to the subject.

Another aspect of the invention relates to multiple doses of the topical avermectin (with or without spinosyn) or ivermectin (with or without spinosad) based formulations of the invention. Multiple applications can include at least one, two, three or four additional dosages beyond the initial dose, with one or possibly two additional doses being preferred.

It is known in the art that similar treatment regimes are presently utilized to treat not only head lice infestations, but also infestations of pubic lice or body lice. Thus, it will be evident that the avermectin-containing formulations of the invention will also be effective in treating not only head lice, but also infestations of the human body of pubic lice and body lice. The formulations of the invention may be used to treat a pubic lice and/or body lice infestation. The core components of the formulation can be altered to provide for a formulation with the consistency of a cream rinse or lotion that may be applied to the affected areas, left on for a period of time as contemplated for treatment of head lice, and then rinsed off. Multiple dosing may also occur as contemplated herein for treating head lice with a formulation as disclosed herein.

The following examples are provided to describe exemplary aspects of the invention in greater detail. They are intended to illustrate, not to limit, the invention.

### Example 1

### Formulation of an Ivermectin Shampoo Conditioner

A formulation comprising ivermectin and/or spinosad as a topical shampoo-conditioner for eliminating treatment resistant lice may be prepared as follows:

The ivermectin is weighed and pre-dissolved in a vessel containing a water-miscible surface active agent, hereinafter Phase A, i.e., 15.00% w/v of polysorbate 80. Phase A is heated, with mixing, at a constant temperature of 65° C. until the ivermectin is completely dissolved in the surface active agent. Phase A is then poured into vessel containing Phase B, which consists of suspending agents, preservatives, non-ionic surfactants, humectants, and a conditioner agent.

Phase B consists of 27.25% w/v of olive oil, 2.00% w/v of shea butter, 8.00% w/v of lanolin alcohol, 3.00% w/v of cyclomethicone, 0.50% w/v of sorbitan triesterate, 0.20% w/v of methylparaben and 0.05% w/v of propylparaben. Phases A and B are heated, with mixing, at a constant temperature of 85° C. until all ingredients are dissolved and/or melted. Concurrently Phase C, consisting of water buffered with 0.055% w/v citric acid and 1.099: w/v sodium citrate, is heated at a constant temperature of 85° C. With vigorous mixing Phases A and B are slowly added to Phase C. Mixing is continued at or near room temperature until a uniform, homogenous mixture is formed, which is then subsequently packaged.

### Example 2

### Formulation of an Ivermectin Emulsion

Ivermectin is weighed and dissolved in the water-miscible surface active agents Crodalan AWS and oleyl alcohol and the resulting solution (Phase A) is heated to 60-65° C. Suspending agents, non-ionic surfactants, and a conditioner agent, consisting respectively of 27.25% w/v of olive oil and 2.00% w/v of Shea butter, 8.00% w/v of lanolin alcohol and 0.50% w/v of sorbitan triesterate, and 3.00% w/v of cyclomethicone (Phase B) are combined and heated with mixing to 75-80° C. and the dissolution of all ingredients is confirmed. Concurrently Phase C, consisting of water buffered with 0.055% w/v citric acid and 1.099% w/v sodium citrate and methyl and isopropyl parabens, is thoroughly mixed and heated to between 75-80 deg C. Phase A is premixed with Phase B, and with vigorous mixing, Phase C is subsequently added. Mixing is continued until a uniform, homogenous blend is formed. The temperature of the blend is brought down to room temperature under continued mixing, after which time the compounded formulation is packaged.

### Example 3

### USP Antimicrobial Effectiveness Test

Topical ivermectin emulsions comprising solubilizers, non-ionic surfactants, and suspending agents were prepared and screened for antimicrobial efficacy according to the U.S. Pharmacopoeia (USP) Chapter 31, Section 51 (2008), incorporated by reference herein. Ten emulsions were formulated to contain paraben preservatives with or without citric acid and sodium citrate as shown in Table 2 below. The control formulation did not contain citric acid, or sodium citrate.

TABLE 2
Ivermectin formulations

| | Control | 1 | 6 | 8 | 9 | 12 | 14 | 17 | 20 | 25 | 26 |
|---|---|---|---|---|---|---|---|---|---|---|---|
| Ingredient | % w/w | % w/w | % w/w | % w/w | % w/w | % w/w | % w/w | % w/w | % w/w | % w/w | % w/w |
| Purified Water USP | 33.00 | 31.85 | 26.85 | 31.75 | 32.85 | 31.75 | 31.65 | 31.70 | 32.90 | 30.85 | 30.85 |
| Ivermectin | 0.50 | 0.50 | 0.50 | 0.50 | 0.50 | 0.50 | 0.50 | 0.50 | 0.50 | 0.50 | 0.50 |
| Crodalan AWS | 15.00 | 15.00 | 15.00 | 15.00 | 15.00 | 15.00 | 15.00 | 15.00 | 15.00 | 15.00 | 15.00 |
| Oleyl Alcohol NF | 10.00 | 10.00 | 10.00 | 10.00 | 10.00 | 10.00 | 10.00 | 10.00 | 10.00 | 10.00 | 10.00 |
| Lanolin Alcohol NF | 8.00 | 8.00 | 8.00 | 8.00 | 8.00 | 8.00 | 8.00 | 8.00 | 8.00 | 8.00 | 8.00 |
| Olive Oil NF | 27.75 | 27.75 | 27.75 | 27.75 | 27.75 | 27.75 | 27.75 | 27.75 | 27.75 | 27.75 | 27.75 |
| Shea Butter | 2.00 | 2.00 | 2.00 | 2.00 | 2.00 | 2.00 | 2.00 | 2.00 | 2.00 | 2.00 | 2.00 |
| Sorbitan Tristearate | 0.50 | 0.50 | 0.50 | 0.50 | 0.50 | 0.50 | 0.50 | 0.50 | 0.50 | 0.50 | 0.50 |
| Cyclomethicone NF | 3.00 | 3.00 | 3.00 | 3.00 | 3.00 | 3.00 | 3.00 | 3.00 | 3.00 | 3.00 | 3.00 |
| Methylparaben NF | 0.20 | 0.20 | 0.20 | 0.25 | 0.25 | 0.20 | 0.25 | 0.20 | 0.20 | 0.20 | 0.20 |
| Propylparaben NF | 0.05 | 0.05 | 0.05 | 0.10 | 0.10 | 0.05 | 0.10 | 0.05 | 0.05 | 0.05 | 0.05 |
| Propylene Glycol | x | | | | | | | | | | 1.00 |
| USP | | | | | | | | | | | |
| Butylparaben NF | x | | | | 0.05 | | | | | | |
| Citric Acid USP | x | 0.055 | 0.055 | 0.055 | | 0.055 | 0.055 | 0.055 | | 0.055 | 0.055 |
| Sodium Citrate USP | x | 1.099 | 1.099 | 1.099 | | 1.099 | 1.099 | 1.099 | | 1.099 | 1.099 |
| Na₂ EDTA | x | | | | | 0.10 | 0.10 | | | | |
| Sodium Benzoate | x | | | | | | | 0.15 | | | |
| Germall Plus - | x | | | | | | | | 0.10 | | |
| Powder | | | | | | | | | | | |
| Glycerin USP | x | | 5.00 | | | | | | | 1.00 | |
| total | 100.00 | 100.00 | 100.00 | 100.00 | 100.00 | 100.00 | 100.00 | 100.00 | 100.00 | 100.00 | 100.00 |

Under the USP, compendial articles are divided into four categories, see Table 3. The criteria of antimicrobial effectiveness for these products are a function of the route of administration.

TABLE 3
Compendial Product Categories

| Category | Product Description |
|---|---|
| 1 | Injections, other parenterals including |
| | emulsions, otic products, sterile nasal |
| | products, and ophthalmic products made |
| | with aqueous bases or vehicles. |
| 2 | Topically used products made with aqueous |
| | bases or vehicles, nonsterile nasal products, |
| | and emulsions, including those applied |
| | to mucous membranes. |
| 3 | Oral products other than antacids, made with |
| | aqueous bases or vehicles. |
| 4 | Antacids made with an aqueous base. |

The USP test requires cultures of the following microorganisms: Candida albicans (ATCC No. 10231), Aspergillus niger (ATCC No. 16404), Escherichia coli (ATCC No. 8739), Pseudomonas aeruginosa (ATCC No. 9027), and Staphylococcus aureus (ATCC No. 6538), and the microorganisms used in the test must not be more than five passages removed from the original ATCC culture.

Each container was inoculated with one of the prepared and standardized organisms, and mixed. The volume of the suspension inoculum used was between 0.5% and 1.0% of the volume of the formulation. The concentration of test microorganisms added to the formulation after inoculation was between 1×10⁵ and 1×10⁶ cfu per mL of the product.

The inoculated containers were incubated according to the conditions set forth in Table 4. Sample each container at the appropriate intervals specified in Table 5 below. The number of cfu present in each test preparation were determined by the plate count procedure for the applicable intervals. Using calculated concentrations of cfu per mL present at the start of the test, the change in log₁₀ values of the concentration of cfu per mL was calculated for each microorganism at the applicable test intervals, and the changes were expressed in terms of log reductions.

TABLE 4
Culture Conditions for Inoculum Preparation

| | | | Inoculum | Microbial |
|---|---|---|---|---|
| | | Incubation | Incubation | Recovery |
| Organism | Suitable Medium | Temperature | Time | Incubation Time |
| Escherichia coli | Soybean-Casein Digest | 32.5 ± 2.5° | 18 to 24 | 3 to 5 days |
| (ATCC No. 8739) | Broth; | | hours | |
| | Soybean-Casein Digest | | | |
| | Agar | | | |
| Pseudomonas | Soybean-Casein Digest | 32.5 ± 2.5° | 18 to 24 | 3 to 5 days |
| aeruginosa | Broth; | | hours | |
| (ATCC No. 9027) | Soybean-Casein Digest | | | |
| | Agar | | | |
| Staphylococcus | Soybean-Casein Digest | 32.5 ± 2.5° | 18 to 24 | 3 to 5 days |
| aureus | Broth; | | hours | |
| (ATCC No. 6538) | Soybean-Casein Digest | | | |
| | Agar | | | |
| Candida albicans | Sabouraud Dextrose | 22.5 ± 2.5° | 44 to 52 | 3 to 5 days |
| (ATCC No. 10231) | Agar; | | hours | |
| | Sabouraud Dextrose | | | |
| | Broth | | | |
| Aspergillus niger | Sabouraud Dextrose | 22.5 ± 2.5° | 6 to 10 days | 3 to 7 days |
| (ATCC No. 16404) | Agar; | | | |
| | Sabouraud Dextrose | | | |
| | Broth | | | |

The requirements for antimicrobial effectiveness are met if the criteria specified under Table 5 are met. No increase is defined as not more than 0.5 log 10 units higher than the previous value measured.

TABLE 5
Criteria for Tested Microorganisms
For Category 2 Products

| | |
|---|---|
| Bacteria: | Not less than 2.0 log reduction from the initial count at |
| | 14 days, and no increase from the 14 days' count at 28 days. |
| Yeast and | No increase from the initial calculated count at 14 and 28 |
| Molds: | days. |

The results of the USP antimicrobial efficacy testing of select formulations from Table 2 is provided in Table 6 below. The “Control” formulation (referenced in Table 2) was determined to be inadequate for inoculation as this formulation had visible growth of mold when stored over a period of months under controlled laboratory conditions, even without the specific inoculations (data not shown). Minor variants of the control formulation subsequently were found to routinely fail the USP antimicrobial efficacy test for Candida albicans. (Table 6: Ec=Escherichia coli; Pa=Pseudomonas aeruginosa; Sa=Staphylococcus aureus; Ca=Candida albicans; An=Aspergillus niger).

TABLE 6
Antimicrobial efficacy.

| Formulation | | 7 day | 14 Day | 28 Day |
|---|---|---|---|---|
| No. | Organism/Dil | CFU/g | CFU/g | CFU/g |
| 8 | Ec/−1 | | <10 | <10 |
| | Pa/−1 | | <10 | <10 |
| | Sa/−1 | | <10 | <10 |
| | Ca/−1 | | 235 | <10 |
| | An/−4 | | 6.5 × 10⁵ | 3.0 × 10³ |
| 17 | Ec/−1 | <10 | <10 | <10 |
| | Pa/−1 | <10 | <10 | <10 |
| | Sa/−1 | <10 | <10 | <10 |
| | Ca/−1 | <10 | <10 | <10 |
| | An/−4 | 1.6 × 10⁵ | 1.2 × 10³ | 30 |
| 9 | Ec/−4 | 2.6 × 10⁵ | 4.5 × 10³ | TNTC, TNTC |
| | Pa/−1 | 475 | 3.9 × 10³ | TNTC, TNTC |
| | Sa/−4 | 1.2 × 10⁶ | 3.5 × 10⁴ | 2.6 × 10⁴ |
| | Ca/−4 | 8.4 × 10⁵ | 45 | TNTC, TNTC |
| | An/−4 | 4.4 × 10⁵ | 2.3 × 10⁵ | 9.5 × 10⁴ |
| 20 | Ec/−1 | <10 | <10 | <10 |
| | Pa/−1 | <10 | <10 | <10 |
| | Sa/−1 | <10 | <10 | <10 |
| | Ca/−1 | 80 | <10 | 150 |
| | An/−1 | <10 | <10 | <10 |
| 25 | Ec/−1 | <10 | <10 | <10 |
| | Pa/−1 | <10 | <10 | <10 |
| | Sa/−1 | <10 | <10 | <10 |
| | Ca/−1 | 285 | <10 | <10 |
| | An/−4 | 6.1 × 10⁵ | 3.8 × 10⁴ | 1.5 × 10⁴ |
| 26 | Ec/−1 | <10 | <10 | <10 |
| | Pa/−1 | <10 | <10 | <10 |
| | Sa/−1 | <10 | <10 | <10 |
| | Ca/−1 | <10 | <10 | <10 |
| | An/−4 | 5.8 × 10⁵ | 1.6 × 10⁴ | 9.5 × 10³ |
| 6 | Ec/−1 | <10 | <10 | <10 |
| | Pa/−1 | <10 | <10 | <10 |
| | Sa/−1 | <10 | <10 | <10 |
| | Ca/−1 | 885 | <10 | <10 |
| | An/−4 | 9.0 × 10⁵ | 1.5 × 10⁵ | 2.4 × 10⁴ |
| 12 | Ec/−1 | <10 | <10 | <10 |
| | Pa/−1 | <10 | <10 | <10 |
| | Sa/−1 | <10 | <10 | <10 |
| | Ca/−1 | 465 | <10 | <10 |
| | An/−4 | 3.8 × 10⁵ | 8.5 × 10³ | 9.5 × 10³ |
| 14 | Ec/−1 | <10 | <10 | <10 |
| | Pa/−1 | <10 | <10 | <10 |
| | Sa/−1 | <10 | <10 | <10 |
| | Ca/−1 | 475 | <10 | <10 |
| | An/−4 | 4.6 × 10⁵ | 5.0 × 10³ | 8.0 × 10³ |
| 1 | Ec/−1 | 3.5 × 10⁵ | <10 | <10 |
| | Pa/−1 | 7.3 × 10⁵ | <10 | <10 |
| | Sa/−1 | 5.5 × 10⁵ | <10 | <10 |
| | Ca/−1 | 1.3 × 10⁵ | <10 | <10 |
| | An/−4 | 5.0 × 10⁵ | 1.2 × 10³ | 3.0 × 10³ |

TNTC: Too numerous to count.

These data show that the overall antimicrobial efficacy in an emulsion formulation can be enhanced by buffering the dispersed aqueous phase of the emulsion to a pH at which the preservative system inhibits growth for a wider variety of microorganism types in the formulation relative to the equivalent formulation in which the aqueous phase is not buffered.

The invention is not limited to the embodiments described and exemplified above, but is capable of variation and modification within the scope and range of equivalents of the appended claims.

What is claimed:

1. A topical formulation that inhibits the growth of microorganisms, comprising an effective amount of an insecticide dissolved in an oil phase comprising a water-miscible or water-soluble surface active agent, a suspending agent, and a non-ionic surfactant, and an aqueous phase comprising one or more preservatives, wherein the aqueous phase is buffered to a pH at which the formulation inhibits a greater amount of microorganism growth relative to the equivalent formulation in which the aqueous phase is not buffered.

2. The topical formulation of claim 1, wherein the insecticide comprises an avermectin.

3. The topical formulation of claim 2, wherein the avermectin is ivermectin, doramectin, selamectin, or abamectin, or combinations thereof.

4. The topical formulation of claim 2, wherein the insecticide further comprises a spinosyn.

5. The topical formulation of claim 4, wherein the spinosyn is selected from the group consisting of spinosyn factors A, B, C, D, E, F, G, H, J, K, L, M, N, 0, P, Q, R, S, T, U, V, W and Y, or combinations thereof.

6. The topical formulation of claim 1, wherein the pH is from about 4.5 to about 6.2.

7. The topical formulation of claim 1, wherein the aqueous phase is buffered with a buffer comprising citric acid and/or sodium citrate.

8. The topical formulation of claim 7, wherein the aqueous phase comprises from about 0.01% to about 0.1% citric acid and from about 1.0% to about 1.25% sodium citrate, by weight of the formulation.

9. The topical formulation of claim 1, wherein the suspending agent comprises olive oil, shea butter, or combinations thereof.

10. The topical formulation of claim 1, wherein the non-ionic surfactant comprises oleyl alcohol, lanolin alcohol, sorbitan tristearate, or combinations thereof.

11. The topical formulation of claim 1, wherein the water-miscible or water soluble surface active agent comprises polysorbate 80, cetyl acetate, acetylated lanolin alcohol, or combinations thereof.

12. The topical formulation of claim 1, wherein the insecticide comprises ivermectin at a concentration of about 0.1 to about 1% by weight of the formulation.

13. The topical formulation of claim 4, wherein the insecticide comprises ivermectin and spinosad at a combined concentration of about 0.1% to about 5% by weight of the formulation.

14. The topical formulation of claim 1, wherein the oil phase comprises 20% to 35% of the suspending agent by weight of the formulation.

15. The topical formulation of claim 1, wherein the oil phase comprises 10% to 20% of the water-miscible or water-soluble surface active agent by weight of the formulation.

16. The topical formulation of claim 1, wherein the oil phase comprises 15% to 45% of the non-ionic surfactant by weight of the formulation.

17. The topical formulation of claim 9, wherein the suspending agent comprises 25% to 28% olive oil by weight of the formulation and 1% to 5% shea butter by weight of the formulation.

18. The topical formulation of claim 1, wherein the preservative comprises methylparaben and propylparaben at a combined concentration of 0.01 to 2% by weight of the formulation.

19. The topical formulation of claim 1, further comprising a conditioner.

20. The topical formulation of claim 19, wherein the conditioner comprises cyclomethicone at a concentration of 1% to 5% by weight of the formulation.

21. A method for enhancing the resistance of an emulsion formulation to microbial growth, comprising buffering the aqueous phase of the emulsion to a pH at which the formulation inhibits a greater amount of microorganism growth relative to the equivalent formulation in which the aqueous phase is not buffered.

22. The method of claim 21, wherein the formulation is the formulation of claim 1.

23. The method of claim 21, wherein the pH is from about 4.5 to about 6.2.

24. The method of claim 21, wherein the aqueous phase of the emulsion is buffered an effective amount of citric acid and/or sodium citrate.

25. The method of claim 24, wherein said aqueous phase comprises from about 0.01% to about 0.1% citric acid and from about 1% to about 1.25% sodium citrate by weight of the formulation.

26. The method of claim 23, wherein the emulsion comprises ivermectin and the pH inhibits base-catalyzed isomerization of the ivermectin.

27. A method for treating a head lice infestation from a susceptible or treatment-resistant strain of head lice in a subject, comprising topically administering to the subject an effective amount of the formulation of claim 1 for a period of time sufficient to treat the head lice infestation.

28. The method of claim 27, comprising topically administering the formulation to the subject in a single dose.

29. The method of claim 27, wherein the period of time is from about 1 minute to about 60 minutes.

30. The method of claim 27, comprising topically administering the formulation to the subject in two, three, or four doses, wherein the interval between each dose is from 5 to 9 days.
